(19) United States
(12) Patent Application Publication
(10) Pub. No.: US 2020/0306398 A1
(43) Pub. Date: Oct. 1, 2020

(54) SYSTEM AND METHOD FOR TREATMENT/IRRADIATION OF A SURFACE IN A VEHICLE INTERIOR

(71) Applicant: Shanghai Yanfeng Jinqiao Automotive Trim Systems Co. Ltd., Novi, MI (US)

(72) Inventors: Andrew K. Ruiter, Grand Haven, MI (US); Christopher Kring, Zeeland, MI (US); Jiunn Tyng Chen, Holland, MI (US)

(21) Appl. No.: 16/902,669

(22) Filed: Jun. 16, 2020

Related U.S. Application Data
(63) Continuation of application No. PCT/US2018/066184, filed on Dec. 18, 2018.
(60) Provisional application No. 62/616,645, filed on Jan. 12, 2018.

(51) Int. Cl. A61L 2/10; A61L 2/24

(52) U.S. Cl. CPC A61L 2/10; A61L 2202/14; A61L 2202/11; A61L 2/24

(57) ABSTRACT

A system and method of treating a surface in a vehicle interior by radiation/ultraviolet light is disclosed. The method may comprise determining a dose of radiation/ultraviolet light for the surface and directing ultraviolet light to the surface in the dose. The system may comprise a radiation/ultraviolet light source directed by a mechanism and operated by a controller to administer a dose of radiation/ultraviolet light for treatment of the surface. The controller may direct the radiation source on a path to multiple positions/zones of the vehicle interior. The system may comprise at least one apparatus comprising a base and mechanism and ultraviolet light source. Treatment/dose of ultraviolet light may be based on biomatter type potentially on the surface, distance from ultraviolet light source to surface, duration/intensity of applied ultraviolet light and/or other factors/parameters. The dose may be intended to substantially sanitize a surface; different doses may be applied to different surfaces.

### CROSS-REFERENCE TO RELATED APPLICATIONS

The present application is a continuation of PCT/International Patent Application No. PCT/US2018/066184 titled “SYSTEM FOR TREATMENT/IRRADIATION OF A SURFACE IN A VEHICLE INTERIOR” filed Dec. 18, 2018, which claims the benefit of U.S. Provisional Patent Application No. 62/616,645 titled “VEHICLE INTERIOR COMPONENT” filed Jan. 12, 2018.

The present application claims priority to and incorporates by reference in full the following patent applications: (a) U.S. Provisional Patent Application No. 62/616,645 titled “VEHICLE INTERIOR COMPONENT” filed Jan. 12, 2018; (b) PCT/International Patent Application No. PCT/US2018/066184 titled “SYSTEM FOR TREATMENT/IRRADIATION OF A SURFACE IN A VEHICLE INTERIOR” filed Dec. 18, 2018.

### FIELD

The present invention relates to a system for treatment/irradiation of a surface in a vehicle interior.

The present invention also relates to a method for treatment/irradiation of a surface in a vehicle interior.

The present invention further relates to a system and method of treating at least one surface in an interior of a vehicle by irradiation from a radiation source such as an ultraviolet light source.

### BACKGROUND

It is known to treat a surface of an object with irradiation such as from an ultraviolet light source.

It would be advantageous to provide an improved system and/or method for treatment/irradiation of a surface in a vehicle interior.

It would be advantageous to provide an improved system and/or method to treat a surface/object in a vehicle interior with radiation such as from an ultraviolet light source.

### SUMMARY

The present invention relates to a method of treating at least one surface in a vehicle interior of a vehicle by ultraviolet light from an apparatus comprising the steps of selecting a surface of the at least one surface for ultraviolet light, determining a dose of ultraviolet light for the surface and directing ultraviolet light from the ultraviolet light source to the surface for the dose. The apparatus may comprise an ultraviolet light source directed by a mechanism; the apparatus may be operated by a controller; the mechanism may be operated to direct movement of the ultraviolet light source to administer the dose of ultraviolet light within the vehicle interior. The controller may comprise a control program. The method may comprise the steps of obtaining data for a controller and operating the apparatus to direct ultraviolet light within the interior of the vehicle according to the control program for the controller. The controller may be configured to operate with data comprising configuration of the vehicle interior. The dose of the ultraviolet light may comprise at least one of (a) a target dose based on a type of biomatter potentially on the surface; (b) a dose of ultraviolet light for the at least one surface is selected based on biomatter expected to be probable on the at least one surface; (c) a determined dose based on the distance from the ultraviolet light source to the surface in the vehicle interior; (d) a duration of ultraviolet light comprising a minimum required duration based on a type of biomatter potentially on the surface; (e) a duration based on the distance from the ultraviolet light source to the surface in the vehicle interior; (f) an intensity at a wavelength of ultraviolet light from the ultraviolet light source; (g) a dose intended to substantially sanitize the surface; (h) a first dose for the first surface and a second dose for the second surface. The step of selecting a surface for ultraviolet light may comprise selecting a first surface based on a factor; the factor may comprise a determination that the first surface has been touched by an occupant of the vehicle. The method may comprise the step of monitoring status of the vehicle interior; status may comprise at least one of vehicle use and/or vehicle occupancy and/or vehicle data. The at least surface may comprise a first object with at least one surface in a first zone and the at least one surface may comprise a second object with at least one surface in a second zone; and the ultraviolet light source may be configured to provide a first dose for ultraviolet light of the first object in the first zone and a second dose for ultraviolet light of the second object in the second zone. The method may comprise the step of defining a cycle time for ultraviolet light of the vehicle interior by movement of the ultraviolet light source on a path; the at least one surface may comprise surfaces in a first zone and surfaces in a second zone; and the cycle time may comprise a first dose for surfaces in the first zone and a second dose for surfaces in the second zone. The step of determining the dose may be based on parameters; parameters may comprise at least one of area; distance; zone; energy; intensity; duration; cycle; path; speed; selection of surfaces; biomatter; configuration of the vehicle interior. After the vehicle is in use for a duty with a vehicle occupant, the method may comprise the steps of (d) selecting a surface of the vehicle interior for ultraviolet light; (e) determining a dose of ultraviolet light for the surface; (f) directing ultraviolet light from the ultraviolet light source to the surface for the dose; the dose of the ultraviolet light may be determined based on the duty of the vehicle in use; and the surface selected for ultraviolet light may comprise at least one surface expected to be touched by the vehicle occupant of the vehicle in use for the duty. The method may comprise the step of continuing operation of the apparatus according to data for the control program.

The present invention relates to an apparatus configured to treat at least one surface of at least one object in a vehicle interior of a vehicle with ultraviolet light in a dose comprising: a base; a mechanism coupled to the base; an ultraviolet light source configured to be directed by the mechanism to apply ultraviolet light to at least one surface of at least one object in the vehicle interior; a controller configured to operate the mechanism for the ultraviolet light source to administer the dose of ultraviolet light for the at least one surface. The controller may be configured to direct the ultraviolet light source (1) to a first position at a first zone for ultraviolet light for a first duration; (2) to a second position at a second zone for ultraviolet light for a second duration. The ultraviolet light source may comprise at least one of (a) at least one LED lamp; (b) at least one light emitting diode configured to emit light comprising a wavelength of between 200 nm and 300 nm; (c) a light source configured to emit ultraviolet light from at least one light emitting diode; (d) an LED array; (e) a first LED array and a second LED array; (f) a light source comprising a lens; (g) a reflector configured to focus ultraviolet light directed toward a surface in the vehicle interior. Ultraviolet light may be configured to be directed at biomatter; biomatter may comprise at least one of (a) virus and/or (b) germ and/or (c) bacteria; and the dose of ultraviolet light for the at least one surface may be based on biomatter expected to be probable on at least one surface in the vehicle interior. The controller may be configured to direct the ultraviolet light source at least one of (a) along a path within the vehicle interior; (b) along a path from a first position to a second position; (c) along a path within the vehicle interior comprising a generally continuous path in a repeated cycle; (d) to a directed position: (e) from a first position to a second position; (f) to a zone; (g) from a first zone to a second zone. The controller may be configured to control at least one of (a) the dose of ultraviolet light administered to a surface; (b) the intensity of ultraviolet light administered to a surface; (c) the duration of ultraviolet light administered to a surface; (d) position of ultraviolet light administered to a surface; (e) speed of movement of the ultraviolet light source between a first position and a second position; (f) administration of ultraviolet light according to a control program; (g) administration of ultraviolet light according to data; (h) a first dose of ultraviolet light administered to a first surface and a second dose of ultraviolet light administered to a second surface. The controller may be configured to operate with data comprising (a) configuration of the vehicle interior; (b) a path for directed movement of the ultraviolet light source within the vehicle interior to administer ultraviolet light; (c) configuration of the vehicle interior and a path for directed movement of the ultraviolet light source; (d) configuration of at least one surface in the vehicle interior; (e) configuration of at least one surface in the vehicle interior so that ultraviolet light can be directed to the at least one surface for a duration; (f) a path for directed movement of the ultraviolet light source within the vehicle interior comprising application of ultraviolet light to a surface for a duration. The apparatus may comprise a light source configured to emit visible light. The mechanism may comprise a gimbal mechanism; the mechanism may comprise at least one motor; the mechanism may be configured to rotate the ultraviolet light source about a first axis and about a second axis; the first axis is generally orthogonal to the second axis. The controller may be configured to direct the ultraviolet light source from a first position to apply ultraviolet light to a surface of at least one of a seat; a door; a console; a handle; a button; a switch; a seat belt and to a second position to apply ultraviolet light to a surface of another of a seat; a door; a console; a handle; a button; a switch; a seat belt. The controller may be configured to direct the mechanism so that ultraviolet light from the light source is applied to the surface for a predetermined duration based on at least one of (a) biomatter expected to be probable on the surface; (b) the material of the surface; (c) the at least one object providing the surface; (d) a distance of the surface from the ultraviolet light source; (e) expected handling of the surface by at least on occupant of the vehicle; (f) a duty cycle of the vehicle.

The present invention relates to a system configured to treat at least one surface of at least one object in a vehicle interior of a vehicle with ultraviolet light in a dose comprising: at least one apparatus comprising an ultraviolet light source configured to be directed by a mechanism to apply ultraviolet light to at least one surface of at least one object in the vehicle interior; a controller configured to operate the at least one apparatus comprising the ultraviolet light source to administer the dose of ultraviolet light for the at least one surface. The controller may be configured with a control program. The at least one apparatus may comprise a first ultraviolet light source configured to administer a dose of ultraviolet light to a first area of the vehicle interior and a second ultraviolet light source configured to administer a dose of ultraviolet light to a second area of the vehicle interior.

The present invention relates to a system configured to treat at least one surface of at least one object in an interior of a vehicle with irradiation in a dose. The system may comprise a base, a mechanism coupled to the base, a radiation source configured to be directed by the mechanism to irradiate at least one surface of at least one object in the vehicle interior and a controller configured to operate the mechanism for the radiation source to administer the dose of irradiation for the at least one surface. The at least one surface may comprise a surface and the controller may be programmed with configuration of the vehicle interior and a path for directed movement of the radiation source within the vehicle interior to administer irradiation. The path for directed movement of the radiation source within the vehicle interior may comprise application of irradiation to the surface for a duration and directed movement of the radiation source for application or irradiation to another surface of the at least one surface. The controller may be programmed with the configuration of at least one surface in the vehicle interior so that irradiation can be directed to the at least one surface for a duration. The at least one surface may comprise a surface and the controller may be configured to direct the mechanism so that irradiation from the radiation source is applied to the surface for a predetermined duration based on at least one of (a) biomatter expected to be probable on the surface; (b) material of the surface; (c) the at least one object providing the surface; (d) a distance of the surface from the radiation source; (e) expected handling of the surface by at least one occupant of the vehicle; (f) a duty cycle of the vehicle. The radiation source may comprise a light source. The light source may comprise at least one light emitting diode configured to emit light comprising a wavelength of between 200 nm and 300 nm; irradiation may comprise light emitted from the at least one light emitting diode. The system may comprise a lens for the radiation source. The system may comprise a reflector configured to focus irradiation directed toward a surface in the vehicle interior. The at least one object may comprise at least one of a seat, a door, a console, a handle, a button, a switch, a seat belt. The mechanism may comprise a gimbal mechanism. The mechanism may comprise at least one servo motor. The mechanism may be configured to rotate the radiation source about a first axis and about a second axis. The controller may be configured to direct the radiation source along a path within the vehicle interior. The controller may be configured to direct the radiation source from a first position to irradiate a surface of at least one of a seat, a door, a console, a handle, a button, a switch, a seat belt to a second position to irradiate a surface of another of a seat, a door, a console, a handle, a button, a switch, a seat belt. The controller may be configured to direct the radiation source (a) to a first position at a first zone for irradiation for a first duration; (b) to a second position at a second zone for irradiation for a second duration. The first duration may be based on a distance from the radiation source to the first zone, and the second duration may be based on a distance from the radiation source to the second zone. The radiation source may comprise a light source configured to emit ultraviolet light.

The present invention also relates to a method of treating at least one surface in an interior of a vehicle by irradiation from a radiation source directed by a mechanism comprising the steps of selecting a surface of the at least one surface for irradiation, determining a dose of irradiation for the surface, directing irradiation from the radiation source to the surface for the dose. The radiation source may be directed by the mechanism. The dose of the irradiation may be determined based on a distance from the radiation source. The at least one surface may comprise a first surface in a first zone and a second surface in a second zone; the step of directing irradiation may comprise directing a first dose of irradiation for the first surface and directing a second dose of irradiation for the second surface. The mechanism may be operated by a controller configured to direct movement of the radiation source to administer the dose of irradiation for the surface for treatment of biomatter probable on the surface. At least one of (a) the dose; (b) the duration may be based on the distance from the radiation source to the surface in the vehicle interior.

The present invention relates to a component for a vehicle interior to irradiate at least one of a seat, a door, a console, a handle, a button, a switch, a seat belt. The component may comprise an irradiation assembly, a mechanism configured to move the irradiation assembly and a controller configured to control movement of the irradiation assembly. The irradiation assembly may comprise an irradiation source and a reflector configured to focus irradiation from the irradiation source. The irradiation source may comprise a light emitting diode. The light emitting diode may be configured to emit light comprising a wavelength of between 200 nm and 300 nm. The mechanism may be configured to move the irradiation assembly from a first position to irradiate at least one of a seat, a door, a console, a handle, a button, a switch, a seat belt to a second position to irradiate another of a seat, a door, a console, a handle, a button, a switch, a seat belt. The mechanism may be configured to move the irradiation assembly from a first position to irradiate a first portion of at least one of a seat, a door, a console, a handle, a button, a switch, a seat belt to a second position to irradiate a second portion of the seat, door, console, handle, button, switch, seat belt. The mechanism may be configured to rotate the irradiation assembly about a first axis and about a second axis. The first axis may be generally orthogonal to the second axis. The mechanism may comprise a gimbal mechanism. The mechanism may comprise at least one servo motor. The controller may be configured to move the irradiation assembly from a first position to irradiate at least one of a seat, a door, a console, a handle, a button, a switch, a seat belt to a second position to irradiate another of the seat, door, console, handle, button, switch, seat belt. The controller may be configured to move the irradiation assembly from a first position to irradiate a first portion of at least one of a seat, a door, a console, a handle, a button, a switch, a seat belt to a second position to irradiate a second portion of the seat, door, console, handle, button, switch, seat belt. At least one of the controller and the mechanism may be configured to move the irradiation assembly along a path. The path may comprise at least a first position of the irradiation assembly and a second position of the irradiation assembly. The controller may be configured to hold the irradiation assembly in the first position for a first duration of time and in the second position for a second duration of time. When the irradiation assembly is in the first position, the irradiation assembly may irradiate a first surface a first distance away from a point on the irradiation assembly. When the irradiation assembly is in the second position, the irradiation assembly may irradiate a second surface a second distance away from the point on the irradiation assembly. The controller may be configured to hold the irradiation assembly in the first position for a first duration of time and in the second position for a second duration of time. The first duration of time may be set based on the first distance and the second duration of time may be set based on the second distance. The controller may be configured to control the speed of movement of the irradiation assembly between the first position of the irradiation assembly and the second position of the irradiation assembly.

The present invention also relates to a method of irradiating at least one of a seat, a door, a console, a handle, a button, a switch, a seat belt comprising the steps of selecting an irradiation source, selecting surfaces to irradiate, determining a maximum distance from the irradiation source to the surfaces to irradiate, determining a minimum required irradiation, configuring a reflector to focus irradiation from the irradiation source, defining a movement of the irradiation source to provide desired irradiation on the surfaces to irradiate and programming a controller to provide the movement of the irradiation source. The method may comprise the step of determining an angle of reflection for the irradiation source. The method may comprise the step of defining a cycle time to irradiate the surfaces to irradiate. Selecting surfaces to irradiate may comprise determining surfaces touched after irradiation. The method may comprise the step of moving the irradiation source from a first position to irradiate at least one of a seat, a door, a console, a handle, a button, a switch, a seat belt to a second position to irradiate another of the seat, door, console, handle, button, switch, seat belt. The method may comprise the step of moving the irradiation source from a first position to irradiate a first portion of at least one of a seat, a door, a console, a handle, a button, a switch, a seat belt to a second position to irradiate a second portion of the seat, door, console, handle, button, switch, seat belt. The method may comprise the step of holding the irradiation assembly in a first position for a first duration of time and in a second position for a second duration of time.

### FIGURES

FIG. 1A is a schematic perspective view of a vehicle according to an exemplary embodiment.

FIG. 1B is a schematic perspective view of a vehicle showing a vehicle interior according to an exemplary embodiment.

FIG. 1C is a schematic perspective view of a system for treatment/irradiation of a surface in a vehicle interior installed the vehicle interior according to an exemplary embodiment.

FIGS. 2A-2D are schematic perspective views of a system/apparatus for treatment/irradiation of a surface in a vehicle interior according to an exemplary embodiment.

FIG. 2E is a schematic partial front view of a vehicle seat according to an exemplary embodiment.

FIGS. 3A-3C are schematic perspective views of a system for treatment/irradiation of a surface in a vehicle interior according to an exemplary embodiment.

FIGS. 4A and 4B are schematic block diagrams of a system for treatment/irradiation of a surface in a vehicle interior according to an exemplary embodiment.

FIG. 4C is a schematic perspective view of a system/apparatus for treatment/irradiation of a surface in a vehicle interior according to an exemplary embodiment.

FIG. 5A is a schematic exploded perspective view of a system/apparatus for treatment/irradiation of a surface in a vehicle interior according to an exemplary embodiment.

FIGS. 5B and 5C are schematic section views of a system/apparatus for treatment/irradiation of a surface in a vehicle interior according to an exemplary embodiment.

FIGS. 6A and 6B are schematic flow diagrams of methods to operate a system/apparatus for treatment/irradiation of a surface in a vehicle interior according to an exemplary embodiment.

FIG. 7 is a schematic flow diagram of a method to operate a system/apparatus for treatment/irradiation of a surface in a vehicle interior according to an exemplary embodiment.

FIG. 8 is a schematic flow diagram of a method to operate a system/apparatus for treatment/irradiation of a surface in a vehicle interior according to an exemplary embodiment.

FIG. 9 is a schematic flow diagram of a method to operate a system/apparatus for treatment/irradiation of a surface in a vehicle interior according to an exemplary embodiment.

FIG. 10 is a schematic flow diagram of a method to operate a system/apparatus for treatment/irradiation of a surface in a vehicle interior according to an exemplary embodiment.

FIG. 11 is a schematic flow diagram of a method to operate a system/apparatus for treatment/irradiation of a surface in a vehicle interior according to an exemplary embodiment.

### DESCRIPTION

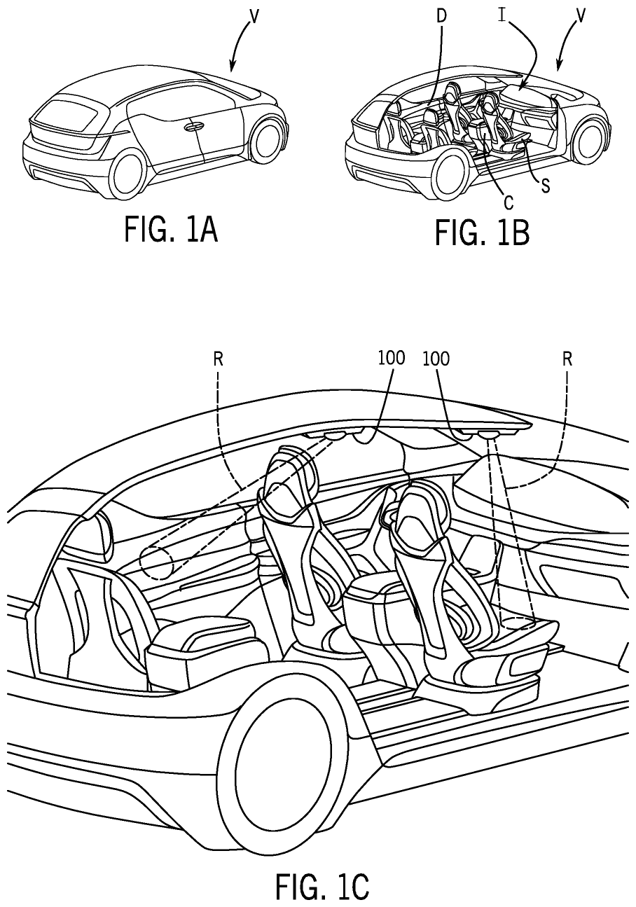

According to an exemplary embodiment as shown schematically in FIGS. 1A and 1B, a vehicle V may comprise an interior I with at least one console C, at least one seat S, and at least one door D. Interior I may comprise at least one of a handle, a button, a switch, a seat belt.

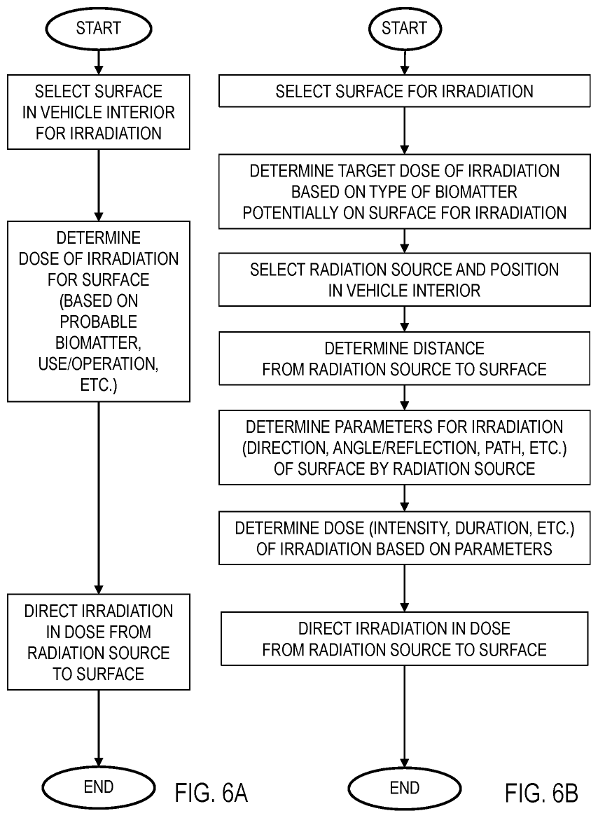

According to an exemplary embodiment as shown schematically in FIG. 1C, a component shown as system/apparatus 100 (e.g. system/apparatus to apply radiation) for a vehicle interior may be configured to irradiate (e.g. to apply radiation R such as UV/ultraviolet light from a UV/ultraviolet light/radiation source) at least one of a seat, a door, a console, a handle, a button, a switch, a seat belt (e.g. application of radiation to surfaces of items, object, etc.). See also FIGS. 6A-6B, 7, 8, 9, 10 and 11 (indicating methods of irradiation for surfaces according to an exemplary embodiment).

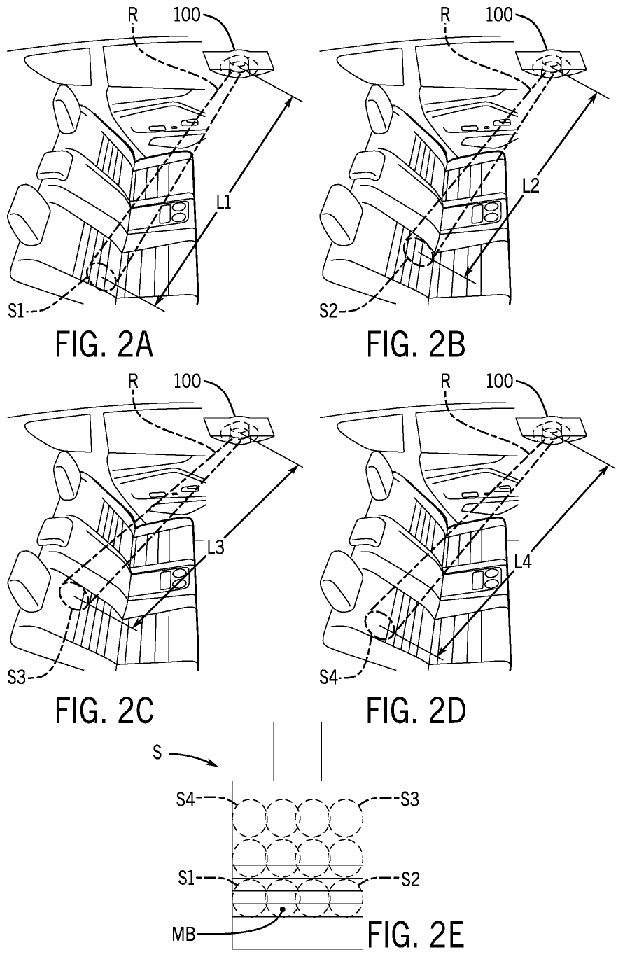

According to an exemplary embodiment, a component 100 for a vehicle interior may be configured to irradiate a first portion S1 of at least one of a seat, a door, a console, a handle, a button, a switch, a seat belt as shown schematically in FIG. 2A and a second portion S2/S3/S4 of the seat, door, console, handle, button, switch, seat belt as shown schematically in FIGS. 2B-2D. See also FIGS. 6A-6B, 7, 8, 9, 10 and 11 (indicating methods of irradiation according to an exemplary embodiment).

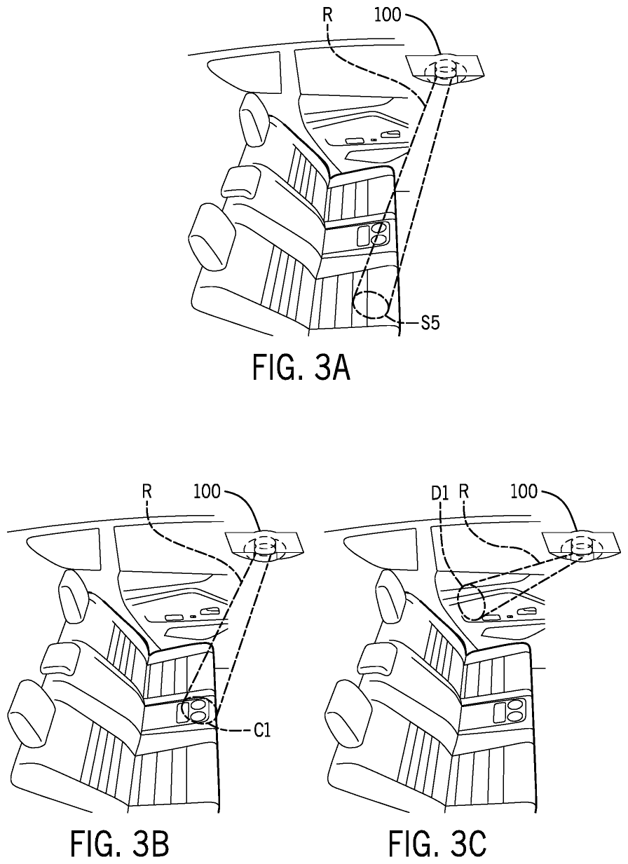

According to an exemplary embodiment, a component 100 for a vehicle interior may be configured to irradiate at least one of a seat, a door, a console, a handle, a button, a switch, a seat belt S1/S2/S3/S4 as shown schematically in FIGS. 2A-2D and another of the seat, door, console, handle, button, switch, seat belt S5 as shown schematically in FIG. 3A. See also FIGS. 6A-6B, 7, 8, 9, 10 and 11 (indicating methods of irradiation according to an exemplary embodiment).

According to an exemplary embodiment, a component 100 for a vehicle interior may be configured to irradiate at least one of a seat, a door, a console, a handle, a button, a switch, a seat belt S1/S2/S3/S4 as shown schematically in FIGS. 2A-2D and another of the seat, door, console, handle, button, switch, seat belt C1 as shown schematically in FIG. 3B. See also FIGS. 6A-6B, 7, 8, 9, 10 and 11 (indicating methods of irradiation according to an exemplary embodiment).

According to an exemplary embodiment, a component 100 for a vehicle interior may be configured to irradiate at least one of a seat, a door, a console, a handle, a button, a switch, a seat belt S1/S2/S3/S4 as shown schematically in FIGS. 2A-2D and another of the seat, door, console, handle, button, switch, seat belt D1 as shown schematically in FIG. 3C. See also FIGS. 6A-6B, 7, 8, 9, 10 and 11 (indicating methods of irradiation according to an exemplary embodiment).

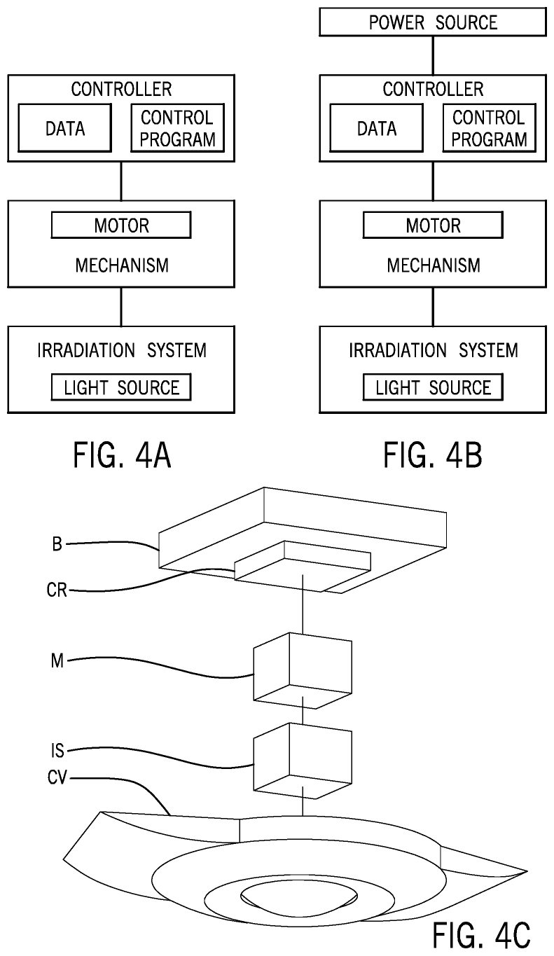

According to an exemplary embodiment as shown schematically in FIGS. 1C, 2A-2E, 3A-3C, 4A-4C and 5A-5C, a system configured to treat a surface of an object in an interior of a vehicle with irradiation in a dose may comprise a base B with a housing/cover CV for a mechanism M configured to direct a radiation source IS (e.g. light source, ultraviolet lamp/array, LED lamp, LED array/matrix, UV lamp, etc.) that will direct the dose/beam of radiation. As shown schematically in FIGS. 4A-4C, the mechanism M is operated by a controller/control system CR and configured to direct the radiation source IS so that the dose/beam of radiation can be applied as treatment (e.g. radiation/irradiation, directed light, etc.) to one or more surfaces in the vehicle interior (e.g. that may contain biomatter MB). See also FIGS. 2A-2E and 3A-3C. As indicated schematically in FIGS. 4A and 4B, the controller (e.g. programmable device, computing device, computer, etc.) operates with a control program and data to operate the mechanism (e.g. motor, etc.) to direct the irradiation system (e.g. radiation source, light source, etc.); power for operation may be supplied by a power source (see FIG. 4B). As shown schematically in FIG. 4C, the radiation source of the system may be concealed behind a door (e.g. retractable door, hinged door, sliding door, etc.) provided by cover CV; the door is closed to conceal the radiation source when the system is not in operation; in operation, the door is opened for the radiation source to direct radiation (e.g. light) toward objects/surfaces in the vehicle interior. See also FIGS. 2A-2D and 3A-3C.

As shown schematically/representationally in FIG. 2E, the system for treatment of a surface of an object in an interior of a vehicle (shown as containing biomatter MB) with irradiation may be configured to treat objects/surfaces in the vehicle interior by directing irradiation/radiation from a radiation source to the objects/surfaces (shown as comprising surfaces S1 through S4 along a path over the object shown as seat S). See also FIGS. 2A-2D and 3A-3C. The system may be configured/programmed to administer the dose of irradiation for treatment of the objects/surfaces based on parameters such as the configuration of the vehicle interior and a path for direction of the radiation source and irradiation/beam to objects/surfaces within the vehicle interior; the controller may be programmed with the a control program and with data based on configuration of objects/surfaces and the radiation source and potential biomatter (e.g. germs, bacteria, virus, microbial material, etc. shown schematically as biomatter MB in FIG. 2E) that may be present on at least one surface in the vehicle interior so that irradiation can be directed to the at least one surface for a dose (e.g. intensity, time/duration, etc.) for treatment (e.g. with intended purpose, efficacy, etc.). See also FIGS. 4A-4B. As indicates schematically, the path for directed movement of the radiation source within the vehicle interior may comprise application of irradiation to the surface of an object for a duration and directed movement of the radiation source for application or irradiation to another surface of an object. See FIGS. 2A-2E and 3A-3C.

According to an exemplary embodiment as shown schematically in FIG. 2E, the object shown as seat S may comprise a surface (e.g. surfaces S1 through S4); the system may configured to direct the radiation source to apply radiation to the surface or surfaces in a path (e.g. from surface S1 to surface S2 to surface S3 to surface S4 and etc.) so that radiation from the radiation source is applied to the surface for a predetermined duration for treatment (e.g. for application to biomatter MB on the surface); the path may be continuous or may be selectively programmed based on parameters/data (e.g. vehicle type/size, use/duty, occupancy, configuration, biomatter/microbial material type, etc.). According to an exemplary embodiment shown in FIGS. 2A-2E, 3A-3C and 4A-4C, the irradiation of the surface by the system may be determined/programmed based on at least one of (a) biomatter expected to be probable on the surface, (b) material of the surface, (c) the at least one object providing the surface, (d) a distance of the surface from the radiation source, (e) expected handling of the surface by at least one occupant of the vehicle, (f) a duty cycle of the vehicle, etc.

According to an exemplary embodiment as shown schematically in FIGS. 1C, 2A-2E, 3A-3C and 5A-5C, a system configured to treat at least one surface of at least one object in an interior of a vehicle with irradiation in a dose may comprise a base, a mechanism coupled to the base, a radiation source configured to be directed by the mechanism to irradiate at least one surface of at least one object in the vehicle interior and a controller configured to operate the mechanism for the radiation source to administer the dose of irradiation for the at least one surface. The at least one surface may comprise a surface and the controller may be programmed with configuration of the vehicle interior and a path for directed movement of the radiation source within the vehicle interior to administer irradiation. The path for directed movement of the radiation source within the vehicle interior may comprise application of irradiation to the surface for a duration and directed movement of the radiation source for application or irradiation to another surface of the at least one surface. The controller may be programmed with the configuration of at least one surface in the vehicle interior so that irradiation can be directed to the at least one surface for a duration. The at least one surface may comprise a surface, and the controller may be configured to direct the mechanism so that irradiation from the radiation source is applied to the surface for a predetermined duration based on at least one of (a) biomatter expected to be probable on the surface; (b) material of the surface; (c) the at least one object providing the surface; (d) a distance of the surface from the radiation source; (e) expected handling of the surface by at least one occupant of the vehicle; (f) a duty cycle of the vehicle. The radiation source may comprise a light source. The light source may comprise at least one LED lamp. The light source may comprise at least one light emitting diode configured to emit light comprising a wavelength of between 200 nm and 300 nm; irradiation may comprise light emitted from the at least one light emitting diode. The light source may comprise a first LED array and a second LED array. The light source may comprise a first LED matrix and a second LED matrix. The system may comprise a lens for the radiation source. The system may comprise a power source for the light source. The system may comprise a reflector configured to focus irradiation directed toward a surface in the vehicle interior. The at least one object may comprise at least one of a seat, a door, a console, a handle, a button, a switch, a seat belt. The mechanism may comprise a gimbal mechanism. The mechanism may comprise at least one servo motor. The mechanism may be configured to rotate the radiation source about a first axis and about a second axis. The first axis may be generally orthogonal to the second axis. The controller may be configured to direct the radiation source along a path within the vehicle interior. The path may comprise a generally continuous path in a repeated cycle. The controller may be configured to direct the radiation source from a first position to irradiate a surface of at least one of a seat, a door, a console, a handle, a button, a switch, a seat belt to a second position to irradiate a surface of another of a seat, a door, a console, a handle, a button, a switch, a seat belt. The controller may be configured to direct the radiation source from a first position to irradiate a surface of at least one of a seat, a door, a console with a first dose of irradiation from the radiation source to a second position to irradiate a surface of at least one of a handle, a button, a switch, a seat belt with a second dose of irradiation from the radiation source. The controller may be configured to direct the radiation source (a) to a first position at a first zone for irradiation for a first duration; (b) to a second position at a second zone for irradiation for a second duration. The first duration may be based on a distance from the radiation source to the first zone. The second duration may be based on a distance from the radiation source to the second zone. The controller may be configured to control a speed of movement of the radiation source between the first position and the second position. The dose of irradiation may comprise an intensity at a wavelength from the radiation source. The dose of irradiation may be intended to at least partially neutralize biomatter on at least one surface; biomatter may comprise at least one of (a) virus; (b) germ; (c) bacteria. The dose of irradiation may be selected based on biomatter expected to be probable on the at least one surface. The radiation source may be configured to emit visible light. The radiation source may comprise a light source configured to emit ultraviolet light.

According to an exemplary embodiment, a method of treating at least one surface in a vehicle interior of a vehicle by irradiation from a radiation source directed by a mechanism may comprise the steps of selecting a surface of the at least one surface for irradiation, determining a dose of irradiation for the surface and directing irradiation from the radiation source to the surface for the dose. The radiation source may be directed by the mechanism. The dose of the irradiation may be determined based on a distance from the radiation source. The at least one surface may comprise a first surface in a first zone and a second surface in a second zone. The step of directing irradiation may comprise directing a first dose of irradiation for the first surface and directing a second dose of irradiation for the second surface. The mechanism may be operated by a controller configured to direct movement of the radiation source to administer the dose of irradiation for the surface for treatment of biomatter probable on the surface. The radiation source may comprise a light source. The radiation source may be configured to emit visible light. The dose of the irradiation may comprise a target dose based on a type of biomatter potentially on the surface. The duration of the irradiation may comprise a minimum required duration based on a type of biomatter potentially on the surface. At least one of (a) the dose; (b) the duration may be based on the distance from the radiation source to the surface in the vehicle interior. The method may further comprise the step of determining an angle of direction for irradiation of the surface by the radiation source. The method may further comprise the step of determining an angle of reflection for irradiation of the surface by the radiation source. The step of selecting a surface for irradiation may comprise selecting a first surface based on a factor; the factor may comprise a determination that the first surface has been touched by an occupant of the vehicle. The vehicle interior may comprise at least one surface on at least one object selected from a seat, a door, a console, a handle, a button, a switch, a seat belt; the radiation source may be directed to irradiate at least one surface of at least one object. The at least one object may comprise a first object with at least one surface in a first zone, and the at least one object may comprise a second object with at least one surface in a second zone; the radiation source may be configured to provide irradiation of the first object in the first zone and irradiation of the second object in the second zone. The method may further comprise the step of defining a cycle time for irradiation of the vehicle interior by movement of the radiation source on a path; the at least one surface may comprise surfaces in a first zone and surfaces in a second zone; the cycle time may comprise a first dose for irradiation of surfaces in the first zone and a second dose for surfaces in the second zone. The path may comprise a repeated path for a cycle of treatment of at least one surface of the vehicle interior with irradiation from the radiation source. The controller may be configured to control speed of movement of the radiation source between the first position and the second position. The dose may comprise an intensity at a wavelength of irradiation from the radiation source. Irradiation may be intended to at least partially neutralize biomatter on at least one surface; biomatter may comprise at least one of (a) virus; (b) germ; (c) bacteria. The dose of irradiation for the at least one surface may be selected based on biomatter expected to be probable on the at least one surface. The dose may comprise a target dose of irradiation based on the biomatter; the dose may be intended to substantially sanitize the surface. The radiation source may comprise a light source configured to emit light in the ultraviolet spectrum. The method may further comprise the steps of (d) selecting a surface of the vehicle interior for irradiation; (e) determining a dose of irradiation for the surface; (f) directing irradiation from the radiation source to the surface for the dose after the vehicle is in use for a duty with a vehicle occupant. The dose of the irradiation may be determined based on the duty of the vehicle in use; the surface selected for irradiation may comprise at least one surface expected to be touched by the vehicle occupant of the vehicle in use for the duty. The surface in the vehicle interior may comprise at least one surface of at least one object selected from a seat, a door, a console, a handle, a button, a switch, a seat belt.

Referring to FIGS. 6A-6B, 7, 8, 9, 10 and 11, a set of methods of operating and using a system configured to treat a surface of an object in an interior of a vehicle with irradiation in a dose is shown schematically and representationally according to an exemplary embodiment.

According to an exemplary embodiment as shown schematically in FIG. 6A, a method for treatment of objects/surfaces in a vehicle interior with irradiation from a radiation source may comprise the steps of selecting a surface in a vehicle interior for irradiation, determining a dose of irradiation for the surface (based on probable biomatter, use/operation, etc.) and directing irradiation in a dose from the radiation source to the surface. See also FIGS. 2A-2D, 2E, 3A-3C and 4A-4C.

According to an exemplary embodiment as shown schematically in FIG. 6B, a method for treatment of objects/surfaces in a vehicle interior with irradiation from a radiation source may comprise the steps of selecting a surface for irradiation, determining a target dose of irradiation based on type of biomatter potentially on the surface for irradiation, selecting a radiation source and position in the vehicle interior, determining a distance from the radiation source to the surface, determining parameters for irradiation (direction, angle/reflection, path, etc.) of the surface by the radiation source, determining a dose (intensity, duration etc.) of irradiation based on parameters and directing irradiation in a dose from the radiation source to the surface. See also FIGS. 2A-2D, 2E, 3A-3C and 4A-4C.

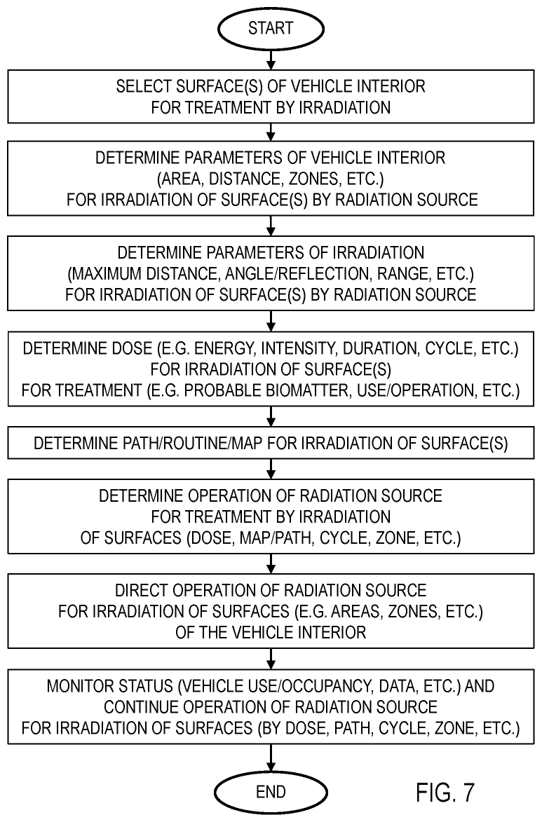

According to an exemplary embodiment as shown schematically in FIG. 7, a method for treatment of objects/surfaces in a vehicle interior with irradiation from a radiation source may comprise the steps of selecting surface(s) of a vehicle interior for treatment by irradiation, determining parameters of the vehicle interior (area, distance, zones, etc.) for irradiation of surface(s) by a radiation source, determining parameters of irradiation (maximum distance, angle/reflection, range, etc.) for irradiation of surface(s) by the radiation source, determining a dose (e.g. energy, intensity, duration, cycle, etc.) for irradiation of surface(s) for treatment (e.g. probable biomatter, use/operation, etc.), determining a path/routine/map for irradiation of surface(s), determining operation of the radiation source for treatment by irradiation of surfaces (dose, map/path, cycle, zone, etc.), directing operation of the radiation source for irradiation of surfaces (e.g. areas, zones, etc.) of the vehicle interior, monitoring status (vehicle use/occupancy, data, etc.) and continuing operation of the radiation source for irradiation of surfaces (e.g. by dose, path, cycle, zone, etc.). See also FIGS. 2A-2D, 2E, 3A-3C and 4A-4C.

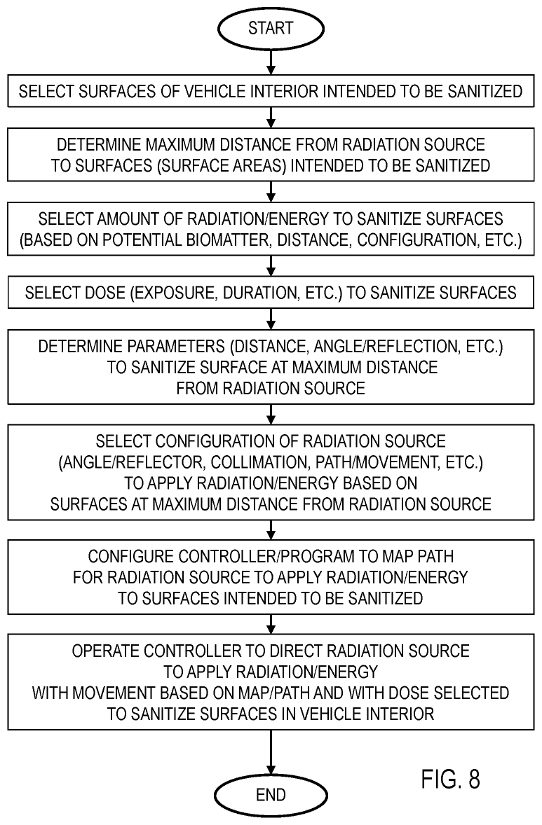

According to an exemplary embodiment as shown schematically in FIG. 8, a method for treatment of objects/surfaces in a vehicle interior with irradiation from a radiation source may comprise the steps of selecting surfaces of a vehicle interior intended to be sanitized, determining a maximum distance from a radiation source to surfaces (surface areas) intended to be sanitized, selecting an amount of radiation/energy to sanitize surfaces (based on potential biomatter, distance, configuration, etc.), selecting a dose (exposure, duration, etc.) to sanitize surfaces, determining parameters (distance, angle/reflection, etc.) to sanitize a surface at the maximum distance from the radiation source, selecting configuration of the radiation source (angle/reflector, collimation, path/movement, etc.) to apply radiation/energy based on surfaces at the maximum distance from radiation source, configuring a controller/program to map a path for the radiation source to apply radiation/energy to surfaces intended to be sanitized, and operating the controller to direct the radiation source to apply radiation/energy with movement based on map/path and with dose selected to sanitize surfaces in the vehicle interior. See also FIGS. 2A-2D, 2E, 3A-3C and 4A-4C.

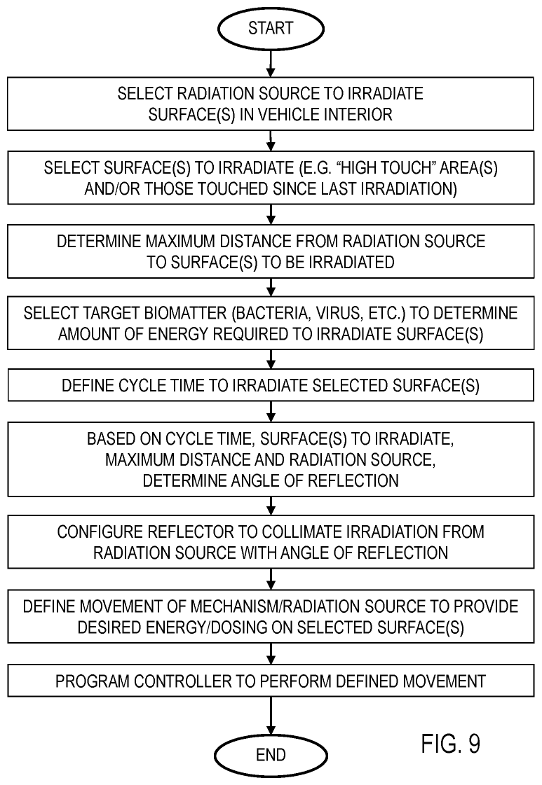

According to an exemplary embodiment as shown schematically in FIG. 9, a method for treatment of objects/surfaces in a vehicle interior with irradiation from a radiation source may comprise the steps of selecting a radiation source to irradiate surfaces in a vehicle interior, selecting surfaces to irradiate (e.g. “high touch” areas and/or those touched since last irradiation), determining a maximum distance from the radiation source to surfaces to be irradiated, selecting target biomatter (bacteria, virus, etc.) to determine amount of energy required to irradiate surfaces, defining a cycle time to irradiate selected surfaces, determining an angle of reflection based on cycle time, surfaces to irradiate, maximum distance and radiation source, configuring a reflector to collimate irradiation from the radiation source with the angle of reflection, defining movement of a mechanism/radiation source to provide desired energy/dosing on selected surfaces and programming a controller to perform defined movement. See also FIGS. 2A-2D, 2E, 3A-3C and 4A-4C.

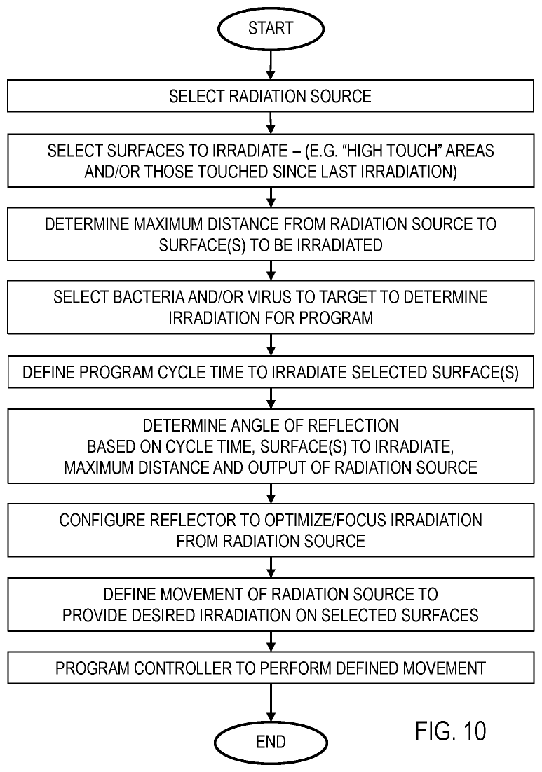

According to an exemplary embodiment as shown schematically in FIG. 10, a method for treatment of objects/surfaces in a vehicle interior with irradiation from a radiation source may comprise the steps of selecting a radiation source, selecting surfaces to irradiate (e.g. “high touch” areas and/or those touched since last irradiation), determining a maximum distance from the radiation source to surfaces to be irradiated, selecting bacteria and/or virus to target to determine irradiation for a program, defining a program cycle time to irradiate selected surfaces, determining an angle of reflection based on cycle time, surfaces to irradiate, maximum distance and output of radiation source, configuring a reflector to optimize/focus irradiation from the radiation source, defining movement of the radiation source to provide desired irradiation on selected surfaces and programming a controller to perform defined movement. See also FIGS. 2A-2D, 2E, 3A-3C and 4A-4C.

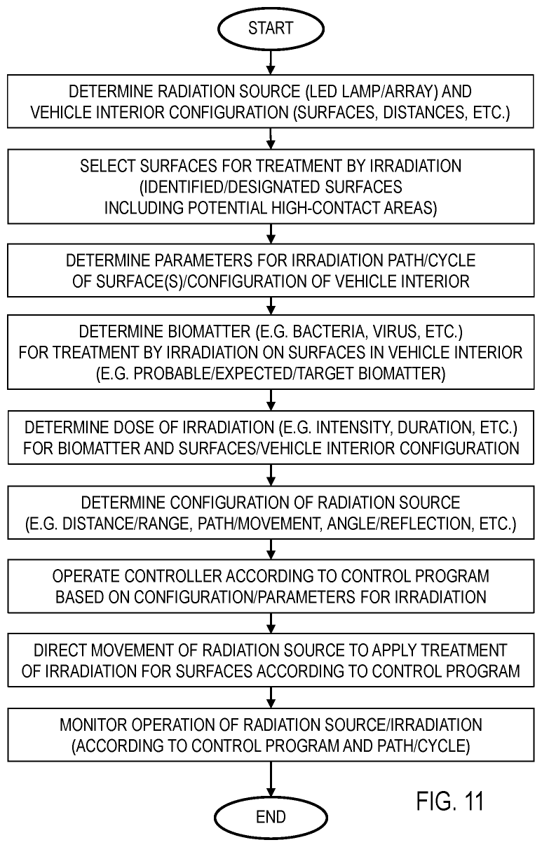

According to an exemplary embodiment as shown schematically in FIG. 11, a method for treatment of objects/surfaces in a vehicle interior with irradiation from a radiation source may comprise the steps of determining a radiation source (led lamp/array) and vehicle interior configuration (surfaces, distances, etc.), selecting surfaces for treatment by irradiation (identified/designated surfaces including potential high-contact areas), determining parameters for irradiation path/cycle of surface(s)/configuration of the vehicle interior, determining biomatter (e.g. bacteria, virus, etc.) for treatment by irradiation on surfaces in the vehicle interior (e.g. probable/expected/target biomatter), determining a dose of irradiation (e.g. intensity, duration, etc.) for biomatter and surfaces/vehicle interior configuration, determining configuration of the radiation source (e.g. distance/range, path/movement, angle/reflection, etc.), operating a controller according to a control program based on configuration/parameters for irradiation, directing movement of the radiation source to apply treatment of irradiation for surfaces according to the control program and monitoring operation of the radiation source/irradiation (according to control program and path/cycle). See also FIGS. 2A-2D, 2E, 3A-3C and 4A-4C.

As indicated schematically in FIGS. 6A-6B, 7, 8, 9, 10 and 11, the methods of operating and using a system configured to treat a surface of an object in an interior of a vehicle with irradiation in a dose may be configured with a wide variety of steps and sequences (e.g. programmed/determined according to the parameters of use, configuration of the interior, other data, etc.) according to an exemplary embodiment.

### EXEMPLARY EMBODIMENTS

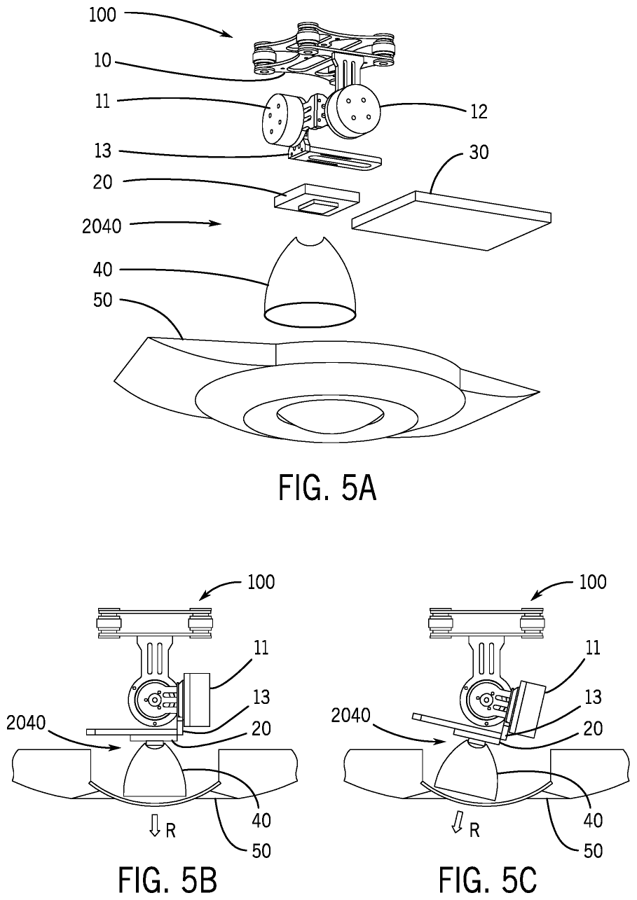

According to an exemplary embodiment as shown schematically in FIG. 5A, a component 100 (e.g. system/apparatus) for a vehicle interior may comprise an irradiation assembly 2040, a mechanism 10 configured to move irradiation assembly 2040 and a controller 30 configured to control movement of irradiation assembly 2040. Component 100 may comprise a housing 50. Component 100 may be configured to irradiate at least one of a seat, a door, a console, a handle, a button, a switch, a seat belt. Irradiation assembly 2040 may comprise an irradiation source 20 and a reflector 40 configured to focus irradiation from irradiation source 20. Irradiation source 20 may comprise a light emitting diode. The light emitting diode may be configured to emit light comprising a wavelength of between 200 nm and 300 nm. Mechanism 10 may comprise a gimbal mechanism. Mechanism 10 may comprise at least one servo motor 11/12. Controller 30 may be configured to move irradiation assembly 2040 from a first position as shown schematically in FIG. 5B to irradiate at least one of a seat, a door, a console, a handle, a button, a switch, a seat belt to a second position as shown schematically in FIG. 5C to irradiate another of the seat, door, console, handle, button, switch, seat belt. (See also FIGS. 2A-2E and 3A-3C.) Controller 30 may be configured to move irradiation assembly 2040 from a first position to irradiate a first portion of at least one of a seat, a door, a console, a handle, a button, a switch, a seat belt to a second position to irradiate a second portion of the seat, door, console, handle, button, switch, seat belt. (See also FIGS. 2A-2E.) At least one of controller 30 and mechanism 10 may be configured to move irradiation assembly 2040 along a path. The path may comprise at least a first position of irradiation assembly 2040 and a second position of irradiation assembly 2040. (See FIGS. 2A-2E.) Controller 30 may be configured to hold irradiation assembly 2040 in the first position for a first duration of time and in the second position for a second duration of time. (See FIGS. 2A-2E.) When irradiation assembly 2040 is in the first position, irradiation assembly 2040 may irradiate a first surface a first distance away from a point on irradiation assembly 2040. When irradiation assembly 2040 is in the second position, irradiation assembly 2040 may irradiate a second surface a second distance away from the point on irradiation assembly 2040. Controller 30 may be configured to hold irradiation assembly 2040 in the first position for a first duration of time and in the second position for a second duration of time. The first duration of time may be set based on the first distance and the second duration of time may be set based on the second distance. Controller 30 may be configured to control the speed of movement of irradiation assembly 2040 between the first position of irradiation assembly 2040 and the second position of irradiation assembly 2040. See also FIGS. 6A-6B, 7, 8, 9, 10 and 11 (indicating methods of irradiation according to an exemplary embodiment).

According to an exemplary embodiment as shown schematically in FIGS. 5B-5C, mechanism 10 may be configured to move irradiation assembly 2040 from a first position as shown schematically in FIG. 5B to irradiate at least one of a seat, a door, a console, a handle, a button, a switch, a seat belt to a second position as shown schematically in FIG. 5C to irradiate another of a seat, a door, a console, a handle, a button, a switch, a seat belt. (See also FIGS. 2A-2E.) Mechanism 10 may be configured to move irradiation assembly 2040 from a first position as shown schematically in FIG. 5B to irradiate a first portion of at least one of a seat, a door, a console, a handle, a button, a switch, a seat belt to a second position as shown schematically in FIG. 5C to irradiate a second portion of the seat, door, console, handle, button, switch, seat belt. (See also FIGS. 2A-2E.) Mechanism 10 may be configured to rotate irradiation assembly 2040 about a first axis and about a second axis. The first axis may be generally orthogonal to the second axis. See also FIGS. 6A-6B, 7, 8, 9, 10 and 11 (indicating methods of irradiation according to an exemplary embodiment).

According to an exemplary embodiment, component 100 may be configured to irradiate (treat/sanitize) interior surfaces of a vehicle by using UV-C light (e.g. from a light emitting diode or irradiation source 20) with intent to target/kill germs and/or bacteria (e.g. microbial material, biomatter, etc. as indicated in FIG. 2E). Using UV-C light emitted by a light emitting diode may be preferable to using mercury-based bulbs, which are comparatively fragile and require a higher amount of energy to kill (e.g. neutralize, etc.) selected virus(es), germs and/or bacteria. Given a specific LED with a known intensity or power output, time required to kill (e.g. neutralize, etc.) selected virus(es), germs and/or bacteria at a distance from LED may be calculated using the formula:

UV Dose=UV Intensity (I)×Exposure Time (t)

Conventional dosing tables listing doses required for selected bacteria and viruses may be consulted in order to select the UV Dose. For example, a dose of 3000 mWs/cm2 may be selected to target E. coli (Escherichia coli) and Staphylococcus aureus.

According to an exemplary embodiment, generally the application of irradiation as a treatment of a surface/object in a vehicle interior may be determined as a dose based on application of radiation from a radiation source (e.g. emitter, light source, LED lamp, LED array, multi-LED array, multi-wavelength emitter, etc.) with a specific intensity (e.g. power output/energy, wavelength, composite wavelength, waveform, amplitude, power cycle, constant/variable, mean/average, etc.) for a duration (e.g. time period, exposure period, duty cycle, etc.) targeted generally or for certain/specific types/forms of biomatter (e.g. microbes, microbial material, virus(es), germs and/or bacteria, etc.) on surfaces at a distance from the radiation source in a vehicle configuration and may be calculated generally using a formula:

Radiation Dose=Radiation Intensity (I)×Exposure Time (t)

According to an exemplary embodiment, dose calculations may be performed and stored as data and/or performed by or for the controller/control system (e.g. based on parameters, data, in real time, in advance, etc.); doses intended for selected biomatter, bacteria, viruses etc. may be stored as data for the system. See FIGS. 4A-4B.

Component 100 (e.g. system/apparatus) may be configured to focus irradiation R from irradiation source 20 (e.g. UV/ultraviolet light from a UV/ultraviolet light source/lamp) to concentrate the power output to a defined surface area at a given distance from irradiation source 20 to provide sufficient power to kill (e.g. neutralize, etc.) selected germs and/or bacteria. UV-C LEDs may be configured to provide an amount of power at a given distance from the LED. As distance from the LED increases, power per square unit of surface area decreases. Beyond a given distance, an LED may not be capable to provide sufficient power to kill (e.g. neutralize, etc.) selected virus(es), germs and/or bacteria. Component 100 may comprise reflector 40 to focus irradiation from irradiation source 40 to concentrate the power output to a defined surface area at a given distance from irradiation source 20 to provide sufficient power to kill (e.g. neutralize, etc.) selected germs and/or bacteria. Component 100 may comprise mechanism 10 and controller 30 to control mechanism 10 to move irradiation source 20 and reflector 40 along a path and/or according to a program to provide sufficient dosing at chosen surfaces in a vehicle interior to kill (e.g. neutralize, etc.) targeted bacteria and/or virus(es). A reflector is preferred, as a lens made from conventional a material (e.g. PC, Lexan, acrylic and glass) would absorb irradiation from the irradiation source.

It is important to note that the present inventions (e.g. inventive concepts, etc.) have been described in the specification and/or illustrated in the FIGURES of the present patent document according to exemplary embodiments; the embodiments of the present inventions are presented by way of example only and are not intended as a limitation on the scope of the present inventions. The construction and/or arrangement of the elements of the inventive concepts embodied in the present inventions as described in the specification and/or illustrated in the FIGURES is illustrative only. Although exemplary embodiments of the present inventions have been described in detail in the present patent document, a person of ordinary skill in the art will readily appreciate that equivalents, modifications, variations, etc. of the subject matter of the exemplary embodiments and alternative embodiments are possible and contemplated as being within the scope of the present inventions; all such subject matter (e.g. modifications, variations, embodiments, combinations, equivalents, etc.) is intended to be included within the scope of the present inventions. It should also be noted that various/other modifications, variations, substitutions, equivalents, changes, omissions, etc. may be made in the configuration and/or arrangement of the exemplary embodiments (e.g. in concept, design, structure, apparatus, form, assembly, construction, means, function, system, process/method, steps, sequence of process/method steps, operation, operating conditions, performance, materials, composition, combination, etc.) without departing from the scope of the present inventions; all such subject matter (e.g. modifications, variations, embodiments, combinations, equivalents, etc.) is intended to be included within the scope of the present inventions. The scope of the present inventions is not intended to be limited to the subject matter (e.g. details, structure, functions, materials, acts, steps, sequence, system, result, etc.) described in the specification and/or illustrated in the FIGURES of the present patent document. It is contemplated that the claims of the present patent document will be construed properly to cover the complete scope of the subject matter of the present inventions (e.g. including any and all such modifications, variations, embodiments, combinations, equivalents, etc.); it is to be understood that the terminology used in the present patent document is for the purpose of providing a description of the subject matter of the exemplary embodiments rather than as a limitation on the scope of the present inventions.

It is also important to note that according to exemplary embodiments the present inventions may comprise conventional technology (e.g. as implemented and/or integrated in exemplary embodiments, modifications, variations, combinations, equivalents, etc.) or may comprise any other applicable technology (present and/or future) with suitability and/or capability to perform the functions and processes/operations described in the specification and/or illustrated in the FIGURES. All such technology (e.g. as implemented in embodiments, modifications, variations, combinations, equivalents, etc.) is considered to be within the scope of the present inventions of the present patent document.

The invention claimed is:

1. A method of treating at least one surface in an interior of a vehicle with ultraviolet light from an apparatus comprising the steps of:
(a) selecting a surface of the at least one surface for application of ultraviolet light;
(b) determining a dose of ultraviolet light for the surface; and
(c) directing ultraviolet light from the ultraviolet light source to the surface for the dose;
wherein the apparatus comprises an ultraviolet light source directed by a mechanism;
wherein the apparatus is operated by a controller;
wherein the mechanism is operated to direct movement of the ultraviolet light source to administer the dose of ultraviolet light within the interior of the vehicle.

2. The method of claim 1 wherein the controller comprises a control program and further comprising the steps of obtaining data for a controller and operating the apparatus to direct ultraviolet light within the interior of the vehicle according to the control program for the controller.

3. The method of claim 1 wherein the controller is configured to operate with data comprising configuration of the vehicle interior.

4. The method of claim 1 wherein the dose of the ultraviolet light comprises at least one of (a) a target dose based on a type of biomatter potentially on the surface; (b) a dose of ultraviolet light for the at least one surface is selected based on biomatter expected to be probable on the at least one surface; (c) a determined dose based on the distance from the ultraviolet light source to the surface in the vehicle interior; (d) a duration of ultraviolet light comprising a minimum required duration based on a type of biomatter potentially on the surface; (e) a duration based on the distance from the ultraviolet light source to the surface in the vehicle interior; (f) an intensity at a wavelength of ultraviolet light from the ultraviolet light source; (g) a dose intended to substantially sanitize the surface; (h) a first dose for the first surface and a second dose for the second surface.

5. The method of claim 1 wherein the step of selecting a surface for ultraviolet light comprises selecting a first surface based on a factor; wherein the factor comprises a determination that the first surface has been touched by an occupant of the vehicle.

6. The method of claim 1 further comprising the step of monitoring status of the vehicle interior; wherein status comprises at least one of vehicle use and/or vehicle occupancy and/or vehicle data.

7. The method of claim 1 wherein the at least surface comprises a first object with at least one surface in a first zone and the at least one surface comprises a second object with at least one surface in a second zone; and wherein the ultraviolet light source is configured to provide a first dose for ultraviolet light of the first object in the first zone and a second dose for ultraviolet light of the second object in the second zone.

8. The method of claim 1 further comprising the step of defining a cycle time for ultraviolet light of the vehicle interior by movement of the ultraviolet light source on a path; wherein the at least one surface comprises surfaces in a first zone and surfaces in a second zone; and wherein the cycle time comprises a first dose for surfaces in the first zone and a second dose for surfaces in the second zone.

9. The method of claim 1 wherein the step of determining the dose is based on parameters; wherein parameters comprise at least one of area; distance; zone; energy; intensity; duration; cycle; path; speed; selection of surfaces; biomatter; configuration of the vehicle interior.

10. The method of claim 1 wherein after the vehicle is in use for a duty with a vehicle occupant further comprising the steps of (d) selecting a surface of the vehicle interior for ultraviolet light; (e) determining a dose of ultraviolet light for the surface; (f) directing ultraviolet light from the ultraviolet light source to the surface for the dose; wherein the dose of the ultraviolet light is determined based on the duty of the vehicle in use; and wherein the surface selected for ultraviolet light comprises at least one surface expected to be touched by the vehicle occupant of the vehicle in use for the duty.

11. An apparatus configured to treat at least one surface of at least one object in a vehicle interior of a vehicle with ultraviolet light in a dose comprising:
(a) a base;
(b) a mechanism coupled to the base;
(c) an ultraviolet light source configured to be directed by the mechanism to apply ultraviolet light to at least one surface of at least one object in the vehicle interior; and
(d) a controller configured to operate the mechanism for the ultraviolet light source to administer the dose of ultraviolet light for the at least one surface;
wherein the controller is configured to direct the ultraviolet light source (1) to a first position at a first zone for ultraviolet light for a first duration; (2) to a second position at a second zone for ultraviolet light for a second duration.

12. The apparatus of claim 11 wherein the ultraviolet light source comprises at least one of (a) at least one LED lamp; (b) at least one light emitting diode configured to emit light comprising a wavelength of between 200 nm and 300 nm; (c) a light source configured to emit ultraviolet light from at least one light emitting diode; (d) an LED array; (e) a first LED array and a second LED array; (f) a light source comprising a lens; (g) a reflector configured to focus ultraviolet light directed toward a surface in the vehicle interior.

13. The apparatus of claim 11 wherein ultraviolet light is configured to be directed at biomatter; wherein biomatter comprises at least one of (a) virus and/or (b) germ and/or (c) bacteria; and wherein the dose of ultraviolet light for the at least one surface is based on biomatter expected to be probable on at least one surface in the vehicle interior.

14. The apparatus of claim 11 wherein the controller is configured to direct the ultraviolet light source at least one of (a) along a path within the vehicle interior; (b) along a path from a first position to a second position; (c) along a path within the vehicle interior comprising a generally continuous path in a repeated cycle; (d) to a directed position: (e) from a first position to a second position; (f) to a zone; (g) from a first zone to a second zone.

15. The apparatus of claim 11 wherein the controller is configured to control at least one of (a) the dose of ultraviolet light administered to a surface; (b) the intensity of ultraviolet light administered to a surface; (c) the duration of ultraviolet light administered to a surface; (d) position of ultraviolet light administered to a surface; (e) speed of movement of the ultraviolet light source between a first position and a second position; (f) administration of ultraviolet light according to a control program; (g) administration of ultraviolet light according to data; (h) a first dose of ultraviolet light administered to a first surface and a second dose of ultraviolet light administered to a second surface.

16. The apparatus of claim 11 wherein the controller is configured to operate with data comprising (a) configuration of the vehicle interior; (b) a path for directed movement of the ultraviolet light source within the vehicle interior to administer ultraviolet light; (c) configuration of the vehicle interior and a path for directed movement of the ultraviolet light source; (d) configuration of at least one surface in the vehicle interior; (e) configuration of at least one surface in the vehicle interior so that ultraviolet light can be directed to the at least one surface for a duration; (f) a path for directed movement of the ultraviolet light source within the vehicle interior comprising application of ultraviolet light to a surface for a duration.

17. The apparatus of claim 11 further comprising a light source configured to emit visible light.

18. The apparatus of claim 11 wherein the mechanism comprises a gimbal mechanism; wherein the mechanism comprises at least one motor; wherein the mechanism is configured to rotate the ultraviolet light source about a first axis and about a second axis; wherein the first axis is generally orthogonal to the second axis.

19. The apparatus of claim 11 wherein the controller is configured to direct the ultraviolet light source from a first position to apply ultraviolet light to a surface of at least one of a seat; a door; a console; a handle; a button; a switch; a seat belt and to a second position to apply ultraviolet light to a surface of another of a seat; a door; a console; a handle; a button; a switch; a seat belt.

20. A system configured to treat at least one surface of at least one object in a vehicle interior of a vehicle with ultraviolet light in a dose comprising:
(a) at least one apparatus comprising an ultraviolet light source configured to be directed by a mechanism to apply ultraviolet light to at least one surface of at least one object in the vehicle interior; and
(b) a controller configured to operate the at least one apparatus comprising the ultraviolet light source to administer the dose of ultraviolet light for the at least one surface;
wherein the controller is configured with a control program;
wherein the at least one apparatus comprises a first ultraviolet light source configured to administer a dose of ultraviolet light to a first area of the vehicle interior and a second ultraviolet light source configured to administer a dose of ultraviolet light to a second area of the vehicle interior.
